(19) United States
(12) Patent Application Publication
(10) Pub. No.: US 2005/0272698 A1
(43) Pub. Date: Dec. 8, 2005

(54) Liquid stable composition of oxazaphosphorine with mesna

(76) Inventors: Gautam Vinod Daftary, Maharashtra (IN); Srikanth Annappa Pai, Maharashtra (IN); Sangeeta Hanurmesh Rivankar, Maharashtra (IN); Kumar Subbappa Praveen, Maharashtra (IN)

Correspondence Address: Gary P Oakeson;Thorpe North & Western, PO Box 1219, Sandy, UT, 84091, US

(21) Appl. No.: 10/529,273

(22) PCT Filed: Sep. 4, 2003
(86) PCT No.: PCT/IN03/00298

§ 371 Date: Mar. 25, 2005

(30) Foreign Application Priority Data
Sep. 5, 2002 (IN) 809MUM/02

(51) Int. Cl. A61K 31/724; A61K 31/66

(52) U.S. Cl. 514/58; 514/109

(57) ABSTRACT

A low toxicity, stable oxazaphosphorine containing compositions with mesna for parenteral administration has been described. The process essentially requires addition of an oxazaphosphorine antineoplastic to the aqueous solution of an etherified β-cyclodextrin followed by addition of mesna as such or as an aqueous solution containing optionally, an etherified β-cyclodextrin. Preferably, the oxazaphosphorine antineoplastic is Ifosfamide and the etherified β-cyclodextrin is 2-hydroxypropyl-β-cyclodextrin.

### FIELD OF INVENTION

This invention relates to a process for preparation of low toxicity, stable aqueous ready-to-use oxazaphosphorine-containing compositions comprising an oxazaphosphorine antineoplastic, mesna and an etherified β-cyclodextrin. It has particular, but not exclusive, application to the preparation of compositions containing Ifosfamide, Mesna and 2-hydroxypropyl-β-cyclodextrin (referred to hereinafter as “HPBCD”) suitable for parenteral administration in human beings and other mammals. The invention is more particularly related to a process for preparation of clear aqueous low toxicity compositions of Ifosfamide comprising Ifosfamide, Mesna, HPBCD that are stable over a period of time thereby making them suitable for ready clinical use.

### BACKGROUND OF THE INVENTION

Two main groups of drugs used in the treatment of malignant disease are alkylating agents and the antimetabolites. Ifosfamide and cyclophosphamide are oxazaphosphorine antineoplastic drugs belonging to the alkylating agents group and are being widely used.

Ifosfamide is given intravenously either by injection as a solution diluted to less than 4% or by infusion and is used in the treatment of a variety of solid tumours including those of the cervix, endometrium, lung, ovary, testes and thymus as well as in sarcoma and in the treatment of Burkitts lymphoma.

Ifosfamide is the Approved Name for 3-(2-chloroethyl)-2-[(2-chloroethyl)amino]tetrahydro-2H-1,3,2-oxazaphosphorin-2-oxide and is represented by the formula:

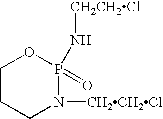

It is a white hygroscopic crystalline powder having a low melting point of 40° C. It also begins to sinter below its melting point. These characteristics of Ifosfamide make it difficult for sterile filling of the dry powder as both temperature and humidity are required to be accurately controlled. Further, as Ifosfamide powder is filled aseptically into sterile containers, maximum precautions are required to maintain sterility of the product.

Ifosfamide powder is freely soluble in water. The aqueous solution is sensitive to changes in pH.

Similar problems are encountered with other oxazaphosphorine antineoplastic, e.g. Cyclophosphamide, which is the Approved Name for 2-[bis(-2-chloroethyl)amino]tetrahydro-2H-1,3,2-oxazophosphorine 2-oxide, represented by the formula:

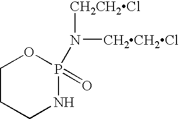

Oxazaphosphorine antineoplastics are toxic to the urinary tract and may involve the kidneys as well as the bladder. Hence it is recommended that they are administered in association with 2-mercaptoethanesulphonates, especially Mesna. Mesna is the Approved Name for sodium-2-mercaptoethanesulphonate and is represented by the formula:
HSCH₂CH₂SO₃⁻Na⁺.

Mesna is highly water soluble. It is used for the prophylaxis of urothelial toxicity in patients being treated with Ifosfamide or Cyclophosphamide. In the kidney Mesna disulfide, the inactive metabolite of Mesna is reduced to free Mesna, which has thiol groups that react with the metabolites of Ifosfamide, and Cyclophosphamide, including acroleil, considered to be responsible for the toxic effects on the bladder.

The intravenous daily dose of Mesna is calculated to equal 60% of the total daily dose of Ifosfamide and is administered as 3 bolus doses given 15 minutes before and 4 and 8 hours after administration of each dose of ifosfamide when the ifosfamide dose is less than 2.5 g/m²/day administered as a short infusion. For use with continuous infusion of Ifosfamide, Mesna may be administered as a bolus dose equal to 20% of the total ifosfamide dose followed by a continuous infusion of mesna equal to 40% of the ifosfamide dose, continuing for 12 to 24 hours after completion of the ifosfamide infusion.

Mesna has also been administered as a continuous infusion at a dose equal to 60% of Ifosfamide dose. No clinical data is available to justify Mesna doses greater than 60% w/w of Ifosfamide for standard doses of Ifosfamide. With high doses in excess of 2.5 g/m² of Ifosfamide, continuous and prolonged Mesna dosage regimen is necessary for maximum protection against urotoxicity.

The disadvantages with the existing commercially available product in powder form is that
1. more than one vial is required to be reconstituted and then diluted to the required concentration as the standard dosage is more than 1 g daily.
2. in high dosage Ifosfamide therapy as high as eight vials of 1 g are required to be reconstituted and diluted to the required concentration.
3. as Mesna is required to be administered along with Ifosfamide, Ifosfamide solution after reconstitution is required to be mixed with Mesna.

Attempts were made by various laboratories/inventors to formulate ready-to-use parenteral solution that would contain Ifosfamide and Mesna to overcome the problem of handling Ifosfamide during reconstitution and during mixing with Mesna.

U.S. Pat. No. 4,959,215 discloses a stable Ifosfamide-Mesna lyophilizate comprising Ifosfamide, 0.05 to 1.0 parts by weight of Mesna and 0.1 to 17 parts by weight of a hexitol prepared by freeze drying an aqueous or aqueous-ethanolic solution of Ifosfamide, Mesna and the hexitol, preferably mannitol. There is no reference to the presence of any cyclodextrin. The lyophilizate is stable physically showing no discolouration. The speed of dissolution is also claimed to markedly higher compared to the dry filled Ifosfamide.

U.S. Pat. No. 4,952,575 discloses a composition comprising 10 to 70 % w/v of an oxazaphosphorine of the formula:

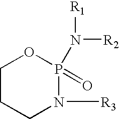

in which at least two of R₁, R₂ and R₃ independently are 2-chloroethyl or 2-methanesulfonyloxyethyl and any remaining R radical is selected from hydrogen, a, methyl and ethyl, dissolved in 80 to 100% v/v of ethanol. Even though the degradation has been shown to be minimal for Ifosfamide, use of solvents in such a high concentration leads to other problems such as volatility, handling during manufacturing miscibility with blood. As ethanol is pharmacologically active, this may also affect the person on administration of alcoholic solution of Ifosfamide.

WO-A-9918973 discloses stable ready-to-use liquid compositions of at least one oxazaphosphorine of the formula:

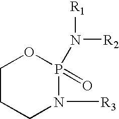

in which R₁, R₂ and R₃ independently are methyl, ethyl, 2-chloroethyl, 2-methanesulfonyloxyethyl or, except for R₃, hydrogen, and at least two of R₁, R₂ and R₃ are 2-chloroethyl and/or 2-methanesulfonyloxyethyl, comprising a physiologically well-tolerated compound which forms chloride ions in aqueous solution. It is independently stated that the composition can include cyclodextrins, preferably α-cyclodextrins, or their ethoxylated derivatives as tonicity adjustment agents and Mesna but there is no exemplification of any composition containing beta cyclodextrin or Mesna.

U.S. Pat. No. 4,879,286 discloses a storage-stable liquid oncolytic formulation of Cyclophosphamide formulated as a ready-to-dilute solution in a carrier comprising 50 to 100% organic polyol selected from propylene glycol, polyethylene glycol and glycerol and 0 to 50% water. The formulation may be used in combination with alcohols such as 10 to 30% of ethanol (based on total weight of the formulation).

U.S. Pat. No. 6,407,079 discloses that the water-solubility and stability of sparingly water-soluble or water-instable drugs are improved by the formation of inclusion compounds with partially etherified β-cyclodextrins of the formula:
(β-CD)OR

in which R are hydroxyalkyl groups with optionally some being alkyl groups,

and having a water-solubility of more than 1.8 g in 100 ml water. Preferably, R are selected from hydroxyethyl, hydroxypropyl or dihydroxypropyl groups. This patent shows use of cyclodextrins for dissolving sparingly water soluble/insoluble drugs and does not indicate its usefulness for water soluble materials like Ifosfamide and Mesna.

U.S. Pat. No. 4,727,064 discloses that lipophilic drugs can be stabilized by solubilizing the drug into an intrinsically amorphous mixture of a water-soluble cyclodextrin derivative to form a solubilized cyclodextin/drug complex and, optionally, freeze-drying or evaporating the resultant solubilized complex to provide a solid cyclodextrin/drug complex in powder form. The exemplified mixtures of cyclodextrin derivatives are obtained by non-selectively alkylating α-, β-, or γ-cyclodextrin using, for example, propylene oxide, glycidol, iodoacetamide, chloroacetate or 2-diethylaminoethylchloride. The cyclodextrins can be substituted by hydroxyalkyl carboxamide, diethylaminoethyl, carboxymethyl or carboxyamidomethyl and exemplified cyclodextrins include hydroxypropyl-β-cyclodexrin. This patent does not suggest use of cyclodextrins for water soluble materials like Ifosfamide and Mesna.

WO-A-0139749 discloses fast dissolving pharmaceutical compositions in solid dosage form with prolonged sweet taste comprising (a) at least one drug, (b) at least one water soluble sugar, (c) at least one non-sugar sweetener in normal fast release form and (d) at least one non-sugar sweetener in a mucoadhesive slow release form. Exemplified drugs include Ifosfamide and Mesna and exemplified mucoadhesive agents include cyclodextrins. There is no exemplification of any composition containing two or more of Ifosfamide, Mesna and a cyclodextrin.

As Mesna is required to be given concurrently with each dose of Ifosfamide, in one aspect of the invention Ifosfamide and Mesna are combined in the same composition to avoid the inconvenience of administering Mesna separately. In another aspect of the invention Ifosfamide and Mesna are combined with HPBCD to give a stable composition so that the product is readily marketable and is convenient to use without the step of reconstitution and less handling. Surprisingly, the process of invention in which Ifosfamide, Mesna and HPBCD are combined has produced a composition having low toxicity also.

The main objective of this invention is thus to develop a process for preparing low toxicity, stable compositions of Ifosfamide comprising Ifosfamide, Mesna, HPBCD, with or without conventional parenteral additives, overcoming all the disadvantages of prior arts and make the composition suitable for parenteral administration in human beings and mammals.

### SUMMARY OF THE INVENTION

Accordingly, the present invention relates to a process for preparation of a low toxicity, stable oxazaphosphorine-containing composition comprising an oxazaphosphorine antineoplastic, mesna and an etherified β-cyclodextrin; the process comprising the steps of:
i) adding the oxazaphosphorine antineoplastic to an aqueous solution of an etherified β-cyclodextrin;
ii) adding mesna as such or as an aqueous solution optionally containing an etherified β-cyclodextrin to the oxazaphosphorine solution of step (i); and
iii) mixng the resultant aqueous solution and, optionally, making up the volume with water.

### DETAILED DESCRIPTION OF EMBODIMENTS OF THE INVENTION

Oxazaphosphorine antineoplastic used in the invention is of the formula:

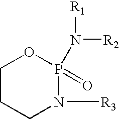

in which at least two of R₁, R₂ and R₃ independently are 2-chloroethyl and the remaining R radical is hydrogen. More preferably, the oxazaphosphorine antineoplastic is Cyclophosphamide (R₁=R₂=chloroethyl & R₃=hydrogen) or, especially, Ifosfamide (R₁=R₃=chloroethyl & R₂=hydrogen).

The etherified β-cyclodextrin preferably has at least some of the hydroxy groups etherified with hydroxyalkyl groups and optionally others etherified with alkyl groups and a water-solubility of more than about 1.8 g/100 ml water. Preferably the hydroxyalkyl groups are hydroxyethyl, dihydroxypropyl or, especially, hydroxypropyl groups and the alkyl groups, if present, are methyl or ethyl groups. The molar substitution (MS) by hydroxyalkyl groups (calculated as moles of alkylating alkylene oxide per anhydroglucose unit) suitably is about 0.05 to about 10, preferably about 0.2 to about 2, and especially about 0.5 to about 1.2.

The oxazaphosphorine antineoplastic content of the composition usually is from about 1 mg/ml to about 1000 mg/ml, preferably from about 25 mg/ml to about 750 mg/ml, and more preferably from about 50 mg/ml to about 500 mg/ml.

The ratio of oxazaphosphorine antineoplastic to mesna is usually in the range of about 20:1 to about 1:2 on a weight basis, preferably in the range of about 10:1 to about 1:1 on a weight basis.

The content of etherified β-cyclodextrin in the composition usually is from about 1% to about 60% w/v, preferably about 2.5% to about 40% w/v, more preferably about 5% to about 20% w/v.

Conventional parenteral additives may be present in the aqueous solution to which the oxazaphosphorine antineoplastic is added and/or in the aqueous solution to which mesna is added. These additives may also be added separately as a solution in water either before adding Mesna to oxazaphosphorine solution or before making up the volume. Such additives can be, for example buffers, isotonic diluents, anticrystallising agents, sequestering agents, or antioxidants as commonly used in aqueous parenteral compositions.

Buffers are selected from pharmaceutically acceptable buffer systems such as, for example, phosphate buffer, citrate buffer, glycine buffer containing any of the commonly used compounds or a mixture of compounds selected from citric acid, sodium citrate, potassium citrate, glycine, phosphoric acid, sodium phosphate, disodium hydrogen phosphate, sodium dihydrogen phosphate, potassium phosphate, dipotassium hydrogen phosphate, potassium dihydrogen phosphate, sodium hydroxide, potassium hydroxide, hydrochloric acid. Preferably the buffer used is a mixture of sodium dihydrogen phosphate and disodium hydrogen phosphate.

The aqueous solutions are mixed, preferably by intimate stirring and the resultant solution is usually sterilized by filtering through a sterilising grade filter. Preferably, the solution is filtered through 2μ and 0.2μ filters successively or just through a 0.21μfilter.

Usually, the filtrate will be aseptically filled into sterile containers such as vials, ampoules, plastic containers and sealing the filled containers.

The invention will now be illustrated by way of Examples. These Examples are by way of illustration only and in no way restrict the scope of the invention.

Ifosfamide used in these Examples was of parenteral grade complying with US Pharmacopoeial specifications. Mesna used in these Examples was of parenteral grade. Hydroxypropyl Beta Cyclodextrin (HPBCD) used was manufactured by Wacker Chemie having degree of substitution per glucose unit by alkyl groups between 0.5 to 1.2. Equipments used were of conventional nature; the entire processing was done in an area with a controlled environment. Water used in these Examples was of parenteral grade complying with “Water for Injection” specifications. All other additives used in these Examples were of parenteral grade.

### EXAMPLE I

| | | |
|---|---|---|
| 1. Ifosfamide | 10 | g |
| 2. Mesna | 2 | g |
| 3. HPBCD | 40 | g |
| 4. Disodium hydrogen phosphate | 0.1 | g |
| 5. Sodium dihydrogen phosphate | 0.06 | g |
| 6. Water | q.s. to 200 | ml |

Weighed quantities of disodium hydrogen phosphate and sodium dihydrogen phosphate were dissolved in 160 ml of water and a weighed quantity of HPBCD was added and dissolved slowly under stirring. The resultant HPBCD solution was divided into two equal parts.

A weighed quantity of Ifosfamide was gradually added under stirring to one part of buffered HPBCD solution and mixed well.

A weighed quantity of Mesna was gradually added under stirring to the remaining part of buffered HPBCD solution and mixed well.

Mesna solution prepared above was added to Ifosfamide solution. The resulting solution was mixed together. The volume was made up to 200 ml with water. The product was filtered through a 0.2μ filter and filled aseptically in sterile glass vials. The glass vials were closed under aseptic conditions with sterile Teflon™ coated rubber bungs and sealed using flip off seals.

The composition obtained in this Example was analysed for Ifosfamide content and Mesna content by high pressure liquid chromatography (HPLC) and was found to contain 52.92 mg/ml of Ifosfamide and 10.2 mg/ml of Mesna. The composition had a pH of 6.86.

### EXAMPLE II

The composition obtained in Example I was subjected to acute toxicity studies in mice. A conventional formulation, Holoxan™ manufactured by M/s. German Remedies was reconstituted as directed by the manufacturer and was used as a control after mixing with Mesna (equivalent to 20% of Ifosfamide content). Both the drug solutions were suitably diluted with 5% Dextrose Injection and administered intravenously. Ifosfamide in the doses of 500 mg/kg, 700 mg/kg and 900 mg/kg body weight was administered in three different groups of animals, each group consisting of eight animals.

The animals were kept under observation for 14 days and mortality recorded at the end of 3 days and 7 days.

It was observed that the LD₅₀ dose was higher for composition of Example I in comparison with the Conventional formulation.

| Composition of Example I | | | Conventional formulation | | |
|---|---|---|---|---|---|
| | Mortality (%) | | | Mortality (%) | |
| Dose (mg) | 3 Days | 7 Days | Dose (mg) | 3 Days | 7 Days |
| 500 | 0 | 0 | 500 | 50 | 75 |
| 700 | 0 | 50 | 700 | 100 | 100 |
| 900 | 75 | 100 | 900 | 100 | 100 |
| LD₅₀ | 700-900 | 700 | LD₅₀ | 500 | <500 |

The above data clearly indicates that composition of Example I is less toxic compared to the Conventional formulation.

### EXAMPLE III

| | | |
|---|---|---|
| 1. Ifosfamide | 10 | g |
| 2. Mesna | 2 | g |
| 3. HPBCD | 20 | g |
| 4. Disodium hydrogen phosphate | 0.1 | g |
| 5. Sodium dihydrogen phosphate | 0.06 | g |
| 6. Water | q.s. to 200 | ml |

Weighed quantities of disodium hydrogen phosphate and sodium dihydrogen phosphate were dissolved in 160 ml of water and a weighed quantity of HPBCD was added and dissolved slowly under stirring.

A weighed quantity of Ifosfamide was gradually added under stirring to the buffered HPBCD solution and mixed for 3 hours.

After 3 hours, a weighed quantity of Mesna was gradually added under stirring to the buffered Ifosfamide solution. The volume was made up to 200 ml with water and filtered through a 0.2μ filter and filled aseptically in sterile glass vials. The glass vials were closed under aseptic conditions with sterile Teflon™ coated rubber bungs and sealed using flip off seals.

### EXAMPLE IV

| | | |
|---|---|---|
| 1. Ifosfamide | 10 | g |
| 2. Mesna | 2 | g |
| 3. HPBCD | 80 | g |
| 4. Disodium hydrogen phosphate | 0.1 | g |
| 5. Sodium dihydrogen phosphate | 0.06 | g |
| 6. Water | q.s. to 200 | ml |

The procedure of Example I was repeated using the components in the amounts set forth above.

### EXAMPLE V

| | | |
|---|---|---|
| 1. Ifosfamide | 10 | g |
| 2. Mesna | 6 | g |
| 3. HPBCD | 20 | g |
| 4. Disodium hydrogen phosphate | 0.1 | g |
| 5. Sodium dihydrogen phosphate | 0.06 | g |
| 6. Water | q.s. to 200 | ml |

The procedure of Example 1 was repeated using the components in the amounts set forth above.

### EXAMPLE VI

| | | |
|---|---|---|
| 1. Ifosfamide | 10 | g |
| 2. Mesna | 16 | g |
| 3. HPBCD | 20 | g |
| 4. Disodium hydrogen phosphate | 0.1 | g |
| 5. Sodium dihydrogen phosphate | 0.06 | g |
| 6. Water | q.s. to 200 | ml |

The procedure of Example I was repeated using the components in the amounts set forth above.

### EXAMPLE VII

| | | |
|---|---|---|
| 1. Ifosfamide | 100 | g |
| 2. Mesna | 20 | g |
| 3. HPBCD | 10 | g |
| 4. Water | q.s. to 200 | ml |

Weighed quantity of HPBCD was dissolved in 20 ml of water. Ifosfamide was added gradually to HPBCD solution under stirring. Mixing was continued till a clear solution was obtained. Mesna was added gradually under stirring to the resultant Ifosfamide solution and mixed well till the entire quantity of Mesna went into solution. Volume was made upto 200 ml with water.

The composition obtained in this Example was analysed for Ifosfamide content and Mesna content and was found to contain 497.88 mg/ml of Ifosfamide and 98.73 mg/ml of Mesna.

### EXAMPLE VIII

| | | |
|---|---|---|
| 1. Ifosfamide | 100 | g |
| 2. Mesna | 60 | g |
| 3. HPBCD | 10 | g |
| 4. Water | q.s. to 200 | ml |

Weighed quantity of HPBCD was dissolved in 20 ml of water. Ifosfamide was added gradually to HPBCD solution under stirring. Mixing was continued till a clear solution was obtained. Mesna was added gradually under stirring to the resultant Ifosfamide solution and mixed well til the entire quantity of Mesna went into solution. Volume was made upto 200 ml with water.

The composition obtained in this Example was analysed for Ifosfamide content and Mesna content and was found to contain 492.02 mg/ml of Ifosfamide and 296.18 mg/ml of Mesna.

### EXAMPLE IX

| | | |
|---|---|---|
| 1. Ifosfamide | 100 | g |
| 2. Mesna | 60 | g |
| 3. HPBCD | 10 | g |
| 4. Disodium hydrogen phosphate | 0.8 | g |
| 5. Sodium dihydrogen phosphate | 0.44 | g |
| 6. Disodium edetate | 0.01 | g |
| 7. Water | q.s. to 200 | ml |

HPBCD was dissolved in 20 ml of water. Ifosfamide was then added gradually to HPBCD solution under stirring. Mixing was continued till a clear solution was obtained. Mesna was added gradually under stirring to the resultant Ifosfamide solution. Disodium edetate, disodium hydrogen phosphate and sodium dihydrogen phosphate were dissolved in 10 ml of water and was added to Ifosfamide-Mesna solution and mixed well. Volume was made upto 200 ml with water.

### EXAMPLE X

| | | |
|---|---|---|
| 1. Ifosfamide | 180 | g |
| 2. Mesna | 36 | g |
| 3. HPBCD | 10 | g |
| 4. Water | q.s. to 200 | ml |

The procedure of Example VIII was repeated using the components in the amounts set forth above.

### EXAMPLE XI

| | | |
|---|---|---|
| 1. Ifosfamide | 10 | g |
| 2. Mesna | 2 | g |
| 3. HPBCD | 20 | g |
| 4. Disodium hydrogen phosphate | 0.1 | g |
| 5. Sodium dihydrogen phosphate | 0.06 | g |
| 6. Water | q.s. to 200 | ml |

Weighed quantities of disodium hydrogen phosphate and sodium dihydrogen phosphate were dissolved in 160 ml of water and a weighed quantity of HPBCD was added and dissolved slowly under stirring. Weighed quantity of Ifosfamide was gradually added under stirring to buffered HPBCD solution and mixed for 3 hours.

Weighed quantity of Mesna was gradually added under stirring to the above Ifosfamide solution and mixed well.

Volume was made up to 200 ml with water. The product was filtered through a 0.2μ filter and filled aseptically in sterile glass vials. The glass vials were closed under aseptic conditions with sterile Teflon™ coated rubber bungs and sealed using flip off seals.

The composition obtained in this Example was analysed for Ifosfamide content and Mesna content and was found to contain 51.2 mg/ml of Ifosfamide and 10 mg/ml of Mesna. The composition had a pH of 7.05.

### EXAMPLE XII

The composition obtained in Example XI along with conventional formulation Holoxan™ manufactured by M/s. German Remedies were subjected to Hemorrhagic cystitis studies in rats to evaluate the bladder toxicity.

Experimental Details are as Follows:

| | |
|---|---|
| Animals used | Wistar rats of either sex. |
| Weight range of animals | 100-150 gm. |
| Number of groups | 5 |
| Number of animals per group | 2 |
| Acclimatization | One week under test conditions |
| | under controlled temperature |
| | and humidity. |

| | |
|---|---|
| Test Materials | Ifosfamide with Mesna Injection |
| Identity | Composition of Example XI |
| Description | Clear colourless solution |
| Route of administration | Intravenous |

| | |
|---|---|
| Comparative material | Holoxan ™ |
| Identity | Ifosfamide injection U.S.P. |
| Lot No. | G 220 |
| Manufacturing Date | October 2001 |
| Expiry Date | September 2003 |
| Description | Dry powder for reconstitution |
| | with water for injection |
| Strength | 40 mg/ml on reconstitution |
| Manufacturer | German Remedies Limited. |
| Route of administration | Intravenous |

Study Designs

Animals were divided into 5 groups and each group comprised two animals. The animals received injections of Ifosfamide formulations as specified in table 1.

TABLE 1
Doses of Ifosfamide Formulations.

Dose (mg/kg body weight)

| Group No. | Formulation | Ifosfamide | Mesna |
|---|---|---|---|
| 1 | Holoxan | 400 | 80 |
| 2 | Holoxan | 500 | 100 |
| 3 | Example XI | 400 | 80 |
| 4 | Example XI | 500 | 100 |
| 5 | Dextrose Inj. | — | — |

All animals received injections via the intravenous route. The animals were sacrificed 24 hours after injection. The urinary bladder of all the animals were collected and was fixed in 10% formalin for 48 hours. Histopathological slides of the organ were prepared and subjected to microscopic examination.

### EVALUATION

Table 2 represents the grading pattern for the hemorrhagic cystitis.

TABLE 2
Grading pattern for hemorrhagic cystitis.

| Grading | Score |
|---|---|
| Normal | 0 (N) |
| Mild hemorrhagic cystitis | 1+ |
| Moderate hemorrhagic cystitis with or without epithelial atypia | 2+ |
| Severe hemorragic cystitis with or without epithelial atypia | 3+ |

### OBSERVATIONS

Table 3 depicts the evaluation results on hemorrhagic cystitis of two formulations of Ifosfamide

TABLE 3
Evaluations of two formulations of Ifosfamide
for hemorrhagic cystitis.

| | |
|---|---|
| Animal | Dose (mg/kg) |

| No. | Formulation | Ifosfamide | Mesna | Score |
|---|---|---|---|---|
| 1. | Holoxan | 400 | 80 | 1+ |
| 2. | Holoxan | 400 | 80 | 1+ |
| 3. | Holoxan | 500 | 100 | 1+ |
| 4. | Holoxan | 500 | 100 | 2+ |
| 5. | Example XI | 400 | 80 | N |
| 6. | Example XI | 400 | 80 | N |
| 7. | Example XI | 500 | 100 | N |
| 8. | Example XI | 500 | 100 | N |
| 9. | Dextrose | — | — | N |
| 10 | Dextrose | — | — | N |

### DISCUSSION

Animals treated with Holoxan showed hemorrhagic cystitis at both doses of 400 mg/kg and 500 mg/kg whereas composition of Example XI did not show hemorrhagic cystitis.

### CONCLUSION

The above findings conclusively proved that the composition of Example XI is less toxic than the conventional formulation Holoxan™

### EXAMPLE XIII

The composition obtained in Example XI was subjected to stability studies. The data is as follows:

| Storage condition | Description | Ifosfamide content | |
|---|---|---|---|
| Initial | Clear, colourless liquid | 51.2 | mg/ml |
| 2° C.-8° C. - 3 M | Clear, colourless liquid | 50.06 | mg/ml |
| 2° C.-8° C. - 6 M | Clear, colourless liquid | 50.33 | mg/ml |

Conclusion:

From the above it is evident that Ifosfamide is stable in the composition obtained in Example XI without undergoing any degradation when stored at 2° C.-8° C. whereas the conventional formulation on reconstitution is reported to be stable for three to six weeks under refrigeration.

The Advantages of the Invention:

1. The composition of present invention is ready-to-use, stable and has low toxicity.
2. Formulating Ifosfamide in aqueous solution with Mesna for parenteral administration provides ease of handling the cytotoxic drug as no reconstitution of the powder formulation is required.
3. The content of Ifosfamide can be increased to as high as 10 g in a 10 ml vial as against 1 to 2 g in a conventional marketed packs. This reduces the number of containers to be handled during administration of the drug.
4. No additional step of mixing with Mesna is required as the composition of present invention is formulated as a solution of Ifosfamide with Mesna.

1. A process for preparation of a low toxicity, stable oxazaphosphorine-containing composition comprising an oxazaphosphorine antineoplastic, mesna and an etherified β-cyclodextrin; the process comprising the steps of:
i) adding the oxazaphosphorine antineoplastic to an aqueous solution of an etherified β-cyclodextrin;
ii) adding mesna as such or as an aqueous solution optionally containing an etherified β-cyclodextrin to the oxazaphosphorine solution of step (i); and
iii) mixing the resultant aqueous solution and, optionally, making up the volume with water.

2. A process as claimed in claim 1, wherein the oxazaphosphorine antineoplastic is of the formula: 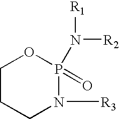
in which at least two of R₁, R₂ and R₃ independently are 2-chloroethyl and the remaining R radical is hydrogen.

3. A process as claimed in claim 2, wherein the oxazaphosphorine antineoplastic is Cyclophosphamide (R₁=R₂=chloroethyl & R₃=hydrogen).

4. A process as claimed in claim 2, wherein the oxazaphosphorine antineoplastic is Ifosfamide (R₁=R₃=chloroethyl & R₂=hydrogen).

5. A process as claimed in any one of the preceding claims, wherein the etherified β-cyclodextrin used is Hydroxypropyl Beta Cyclodextrin (HPBCD).

6. A process as claimed in claim 5, wherein the molar substitution of HPBCD is from about 0.5 to about 1.2.

7. A process as claimed in any one of the preceding claims, wherein the oxazaphosphorine antineoplastic content of the composition is from about 1 mg/ml to about 1000 mg/ml.

8. A process as claimed in claim 7, wherein said oxazaphosphorine antineoplastic content is from about 25 mg/ml to about 750 mg/ml.

9. A process as claimed in claim 8, wherein said oxazaphosphorine antineoplastic content is from about 50 mg/ml to about 500 mg/ml.

10. A process as claimed in claim 9, wherein said oxazaphosphorine antineoplastic content is about 50 mg/ml.

11. A process as claimed in claim 9, wherein said oxazaphosphorine antineoplastic content is about 500 mg/ml.

12. A process as claimed in any one of the preceding claims, wherein the ratio of oxazaphosphorine antineoplastic to mesna is in the range of about 20:1 to about 1:2 on a weight basis.

13. A process as claimed in claim 12, wherein the ratio of oxazaphosphorine antineoplastic to mesna is in the range of about 10:1 to about 1:1 on a weight basis.

14. A process as claimed in claim 13, wherein the ratio of oxazaphosphorine antineoplastic to mesna is 10:2 on a weight basis.

15. A process as claimed in claim 13, wherein the ratio of oxazaphosphorine antineoplastic to mesna is 10:6 on a weight basis.

16. A process as claimed in any one of the preceding claims, wherein the content of etherified β-cyclodextrin in the composition is about 1% to about 60% w/v.

17. A process as claimed in claim 16, wherein said etherified β-cyclodextrin content is about 2.5% to about 40% w/v.

18. A process as claimed in claim 17, wherein said etherified β-cyclodextrin content is about 5% to about 20% w/v.

19. A process as claimed in any one of the preceding claims, wherein one or more conventional parenteral additives are incorporated into the aqueous solution of claim 1 step (i) or claim 1 step (ii) or in water used for making up the volume in claim step (iii).

20. A process as claimed in any one of the preceding claims, wherein said mixture of resultant aqueous solutions is sterilized by filtering through a sterilising grade filter.

21. A process as claimed in claim 20, wherein the filtrate from the sterilising grade filter is aseptically filled into sterile containers and the filled containers are sealed.

22. A process as claimed in claim 1 and substantially as herein before described with reference to any of the Examples.

23. A stable oxazaphosphorine-containing composition obtainable by a process as claimed in any one of the preceding claims.

24. A stable oxazaphosphorine-containing composition prepared by a process as claimed in any one of the preceding claims.

25. The use of a stable oxazaphosphorine-containing composition as defined in claim 23 or claim 24 in the manufacture of a medicament for the treatment of malignant disease.

26. A method of treating a malignant disease comprising administering to a patient suffering said disease an effective amount of a sterile stable oxazaphosphorine-containing composition as defined in claim 23 or claim 24.
